SEQUENCE LISTING (3 sequences)

SEQ ID NO: 1 - DNA, 20 bp, Artificial Sequence
  Oligonucleotide useful as a primer in genotyping Ant in mice.
actcaacctagggccttgtg

SEQ ID NO: 2 - DNA, 21 bp, Artificial Sequence
  Oligonucleotide useful in genotyping Ant in mice.
gggagcattcctgaaaaataa

SEQ ID NO: 3 - DNA, 20 bp, Artificial Sequence
  Oligonucleotide useful in genotyping Ant in mice.
gacttaccctccacgacagc

(19) United States
(12) Patent Application Publication
(10) Pub. No.: US 2005/0091704 A1
(43) Pub. Date: Apr. 28, 2005

(54) Ant2 conditional knockout mouse and methods

(75) Inventors: Douglas C. Wallace, Irvine, CA (US); Grant MacGregor, Irvine, CA (US); Katrina Waymire, Irvine, CA (US); Shawn E. Levy, Brentwood, TN (US); James E. Sligh, Brentwood, TN (US); Jason E. Kokoszka, Frederick, MD (US)

Correspondence Address: GREENLEE WINNER AND SULLIVAN P C, 4875 PEARL EAST CIRCLE, SUITE 200, BOULDER, CO, 80301, US

(73) Assignee: Emory University, Atlanta, GA (US)

(21) Appl. No.: 10/654,628

(22) Filed: Sep. 2, 2003

Related U.S. Application Data
(60) Provisional application No. 60/407,364, filed on Aug. 30, 2002.

(51) Int. Cl. A01K 67/027; C12N 5/06

(52) U.S. Cl. 800/18; 435/354

(57) ABSTRACT

Described are methods for inactivating adenine nucleotide transporter proteins in specific tissues of a transgenic nonhuman animal using a conditional knockin/knockout technology such as the Cre-LoxP, Flip-FLP recombinase, or Tet-on/off technologies. Specifically, the Ant2 gene is functionally inactivated in a mouse in liver, with or without the concurrent inactivation of the Ant1 gene. The result is an animal in which the Ant2 gene and accompanying ANT2 protein is absent in one or more tissues, either in the presence or absence of the Ant1 gene and accompanying ANT1 protein. The resulting animals, cells, mitochondria, and subcelluar fractions such as the mitochondrial permeability transition pore can then be used to identify agents that affect animal and/or subcellular function via a direct or indirect interaction with the ANT2 protein and/or its Ant2 gene.

### CROSS REFERENCE TO RELATED APPLICATIONS

This application claims benefit of U.S. Provisional Application No. 60/407,364, filed Aug. 30, 2002.

### ACKNOWLEDGMENT OF FEDERAL RESEARCH SUPPORT

This invention was made, at least in part, with funding from the National Institutes of Health (Grant Nos. HD36437, NS41850, HL64017, NS21328, and AG13154). Accordingly, the United States Government has certain rights in this invention.

### BACKGROUND OF THE INVENTION

The field of this invention is the area of transgenic animals, mitochondrial energy metabolism and its control and in particular, methods for studying mitochondrial function using a conditional knockout of the Ant2 gene.

Mitochondria are central to both energy metabolism and apoptosis (1,2). ANTs are among the most abundant proteins in the mammalian mitochondrion (3) and are best understood for their role in the exchange of mitochondrial matrix ATP for cytosolic ADP. The ANTs are also thought to be an integral component of the mitochondrial permeability transition pore (mtPTP) (4-6), although this function of the ANTs has been questioned.

Humans have three ANT isoforms: ANT1 which is expressed primarily in the heart and skeletal muscle, ANT2 which is expressed in rapidly growing cells and is inducible, and ANT3 which appears to be constitutively expressed in all tissues (7,8). In contrast, rodents have two ANT isoforms: Ant1, located on chromosome 8, is expressed at high levels in heart, skeletal muscle, and brain; and Ant2, located on the X-chromosome, is expressed in all tissues but skeletal muscle (9). Mouse Ant2 is the ortholog of human ANT2 and seems to combine the functions of human ANT2 and ANT3 (10,11).

Mitochondrial ATP is generated via oxidative phosphorylation through the combined action of five enzyme complexes. Complexes I, II, III, and IV comprise the electron transport chain, which oxidizes electrons donated to complex I from NADH+H⁺ or to complex II from succinate with ½ O₂ to generate H₂O. The energy released by the electron transport chain is used to pump protons across the mitochondrial inner membrane through complexes I, III, and IV creating an electrochemical gradient, ΔP=ΔΨ+ΔpH. The resulting net negative charge in the mitochondrial matrix permits the import of cytosolic Ca⁺⁺ into the mitochondrion through the Ca⁺⁺ uniporter as well as the uptake of lipophilic cations such as tetraphenylphosphonium cation (TPP⁺). The potential energy stored in ΔP is also used by the ATP synthase (complex V) to condense ADP and Pᵢ to make ATP. ΔP and ATP synthesis can be diminished by the expression of uncoupler proteins 1-3 (UCP1-3), which permit protons to flow back across the mitochondrial inner membrane (12,13), thus short circuiting oxidative phosphorylation.

One widely accepted model for the structure of the mtPTP is that the core complex consists of the ANTs, which span the mitochondrial inner membrane, and the voltage dependent anion channels (VDAC, also known as porin), which span the outer membrane. ANT and VDAC associate to form the inner and outer membrane contact sites which may also interact with the pro-apoptotic and anti-apoptotic members of the BCL2 family, cyclophilin D, and the benzodiazepine receptor (6,14).

The mtPTP responds to oxidative stress, excessive Ca⁺⁺ uptake, decreased ΔP or reduced levels of matrix ADP, to form a channel that connects the mitochondrial matrix with the cytosol (15-18). This permits molecules of less than 1500 Da to equilibrate between the mitochondrial matrix and the cytosol, collapsing ΔP and causing the mitochondria to swell.

Apoptosis involves the release of a variety mitochondrial inter-membrane proteins into the cytosol. These include cytochrome c, Smac/DIABLO, apoptosis inducing factor (AIF) and procaspase 9. On entrance into the cytosol, cytochrome c interacts with apoptosis activating factor-1 (Apaf-1), which in the presence of dATP or ATP, leads to recruitment of procaspase-9 to form the “apoptosome.” This activates caspase-9 which in turn can activate additional procaspases resulting in the eventual destruction of the cell (1,19-23).

While the initiation of apoptosis regularly involves the coincident release of cytochrome c and the depolarization of the mitochondrial inner membrane, the relationship of these two events is also in question. One model proposes that the opening of the mtPTP, and the associated swelling of the mitochondrion, results in the disruption of the outer membrane and the release of cytochrome c (23). The other model proposes that aggregates of multi-domain members of the BCL2 family (e.g. BAX, BAK, BOK) form in the outer mitochondrial membrane creating pores that release the cytochrome c (24). The loss of cytochrome c disrupts the electron transport chain, causing its electrons to be redirected into forming reactive oxygen species (ROS), which may initiate the opening of the mtPTP.

In either case, understanding the structure and function of the mtPTP is essential for understanding the molecular events of apoptosis. Since the central function of the mtPTP is the formation of the ion channel between the matrix and the cytosol, it is essential to understand the function of the ANTs in forming this channel. Therefore, we have chosen to investigate the role of the ANTs in the mtPTP and apoptosis, by generating mice in which the Ant1 and Ant2 genes are genetically inactivated. Tissues from these strains are assured of being deficient in the ANTs, and their mitochondria can then be used to analyze the residual function of the mtPTP.

Genetic inactivation of Ant1 (Ant1^{-/-}) resulted in viable mice. However, these animals developed mitochondrial myopathy and severe exercise intolerance along with a hypertrophic cardiomyopathy as young adults (25). Genetic inactivation of the X-linked Ant2 (Ant2^{-/-} or ^{-/Y}) was accomplished using the cre-loxP system to generate tissue-specific ANT2 defects.

Analysis of tissues from these mouse strains demonstrates that the mtPTP in mitochondria containing only ANT1 are much more resistant to Ca⁺⁺ activation than mitochondria with ANT2. Moreover, inactivation of either Ant gene increased the resistance of the mtPTP to Ca⁺⁺ activation. The inactivation of both Ant genes in mouse liver also resulted in increased mtPTP resistance to Ca⁺⁺ activation, but the mtPTP could still be activated by ΔP depolarization or excessive Ca⁺⁺ uptake and initiate the release of cytochrome c. Still, the mtPTPs in ANT-deficient mitochondria lost their ability to be modulated by ANT ligands such as ADP and atractyloside (ATR). Hence, the ANTs are not an essential component of the mitochondrial inner membrane channel of the mtPTP, but are important for its regulation.

### SUMMARY OF THE INVENTION

The present invention provides transgenic mice in which the Ant2 gene is conditionally inactivated (in predetermined tissues). As specifically exemplified herein, a transgenic mouse in which the Ant2 gene is inactivated in the liver has been produced by tissue-specific expression of the cre recombinase in cells in which a critical portion of the Ant2 gene is flanked by lox sites, with the result that the Ant2 gene is functionally inactivated by the Cre-mediated generation of a deletion within the Ant2 gene. Because there is no significant expression of the Ant gene in liver other than Ant2, the liver cells in which the Ant2 gene is functionally inactivated are substantially devoid of any functional Ant2 protein. These mice are useful for studying and identifying compositions which ameliorate the metabolic defects resulting from the liver Ant2 deficiency. Other transgenic mice containing a tissue-specific knock-out of the Ant2 gene can be prepared in a genetic background where the Ant1 gene has also been inactivated.

The present invention further encompasses methods for identifying compositions or environmental conditions (barometric pressure, oxygen availability, temperature, among others) which alter the function of the mitochondrial adenine nucleotide translocators or any of the interactions of same with a cellular component with which they may be interacting, including but not limited to the mtPTP. These methods involve comparisons of the responses of animals, tissues, cells, mitochondria, or cellular fractions with versus without a functional Ant2 protein to various chemical, environmental and physical treatments. These methods can include an Ant1-null mutant in the genetic background of the animals, tissues, cells or subcellular components being examined. The steps in such an analysis could include breeding animals (mice) which lack the Ant2 gene and its accompanying protein in one or more tissues of interests, e.g., the liver. The tissue-specific Ant2-deficiency can be combined with the systemic Ant1-null allele to avoid mistaking the effects of interactions of the ANT1 isoform with the test condition or composition of the ANT2-deficient animals, cells, mitochondria, structures, etc; contacting the Ant2-deficient animals, cells, mitochondria, proteins, or subcellular components such as the mtPTP with the experimental composition or condition (chemical, environment, physical, etc) being tested; comparing the response of the Ant2-deficient as compared to Ant2-containing animals, cell, mitochondria, or subcellular component (eg. mtPTP) to the agent; and identifying test compositions or conditions for which the presence or absence of Ant2 materially alters their action. These data can be used in further methods to identify agents that prove effective treatments for clinical conditions that impinge directly of indirectly on the ANTs, and specifically ANT2.

### BRIEF DESCRIPTION OF THE DRAWINGS

FIG. 1 illustrates preparation of a conditional Ant2-null allele containing loxP sites in embryonic stem cells. A loxP-flanked (floxed, fl) allele of Ant2 was inserted into the ES cell X-chromosome by homologous recombination using a construct in which loxP sequences (triangles) encompassed exons 3 and 4 and a 3′-PGKneo cassette (hatched box) (a). Proper targeting was confirmed by Southern blot (b). The 5′-probe (probe A) detects a 12.8 kb Xba In (x) fragment for the wild type locus and a 6.6 kb fragment for the targeted locus. The 3′-probe (probe B) detects a 7.9 kb DraIII (D) fragment for the wild type and a 13.8 kb fragment for the targeted locus.

FIGS. 2A-2C show abnormal cardiac development in embryonic day 14.5 Ant2-null mice. FIG. 2A: representative control (left) and Ant2-null (right) embryo. Hematoxylin and eosin stained control. FIG. 2B: Ant2-null. FIG. 2C: transverse cardiac section (50×). The Ant2 null heart has a defect in the interventricular septum (asterisk), the ventricle wall (arrowhead), and dilated atria congested with erythrocytes (arrow).

FIGS. 3A-3D illustrate the ultrastructure of cardiomyocytes from the ventricle wall of control and Ant2-null mice. Representative cardiac sections (5,000×) from control (FIG. 3A) and Ant2-null (FIG. 3B) e14.5 embryos illustrating the disruption of muscle fibers (arrowhead), mitochondrial vacuolar inclusions (arrow), and electron dense particles (asterisk). Representative cardiac sections (10,000×) from a control (c) and Ant2-null (FIG. 3D) neonate reveal mitochondria with reduced cristae and vacuolar inclusions (arrowhead).

FIGS. 4A-4D show that Ant1 and Ant2 are expressed in the embryonic heart and cardiomyocyte replication is not inhibited in Ant2-null embryos. In situ hybridization of Ant1 and Ant2-specific riboprobes to e10.5 (FIG. 4A), e12.5 (FIG. 4B), and e14.5 (FIG. 4C) cardiac sections. Top row, wild type hearts. Bottom row Ant1-null (left) or Ant2-null (right) hearts (negative controls) (FIG. 4D) The “% positive BrdU nuclei” is the ratio of nuclei that incorporated BrdU to total nuclei. BrdU incorporation was significantly higher in the ventricle wall (P<0.05), interventricular septum (P<0.05), and trabeculae (P<0.05) in Ant2 null (▪) compared to control (□) embryos (n=3).

FIGS. 5A-5F shows the affect of ANT-deficiency on mtPTP activation, as measured by the amount of Ca⁺⁺ required to elicit permeability transition (PT), detected by irreversible release of TPP⁺. FIGS. 5A-5B: Ca⁺⁺-activation of the mtPTP (FIG. 5A) in skeletal muscle mitochondria in Ant1^{+/+} versus Ant₁^{-/-} mice and (FIG. 5B) in liver mitochondria in Ant2^{+/Y} versus Ant2^{-/Y} mice. FIGS. 5C-5F: Ca⁺⁺-activation of the mtPTP in the liver mitochondria of Ant₁^{-/-}, Ant2^{fl/Y} (ANT-control) and Ant₁^{-/-}, Ant2^{fl/Y}, Alb-Cre (ANT-deficient) mice. FIG. 5C: cytochrome c release after Ca⁺⁺-activation detected by western blot. FIG. 5D: Ca⁺⁺-requirement for mtPTP activation increased three fold by ANT-deficiency (P<0.01) and CsA inhibition (P<0.001, control; P<0.05, ANT-deficient). FIGS. 5E-5F: Altered Ca⁺⁺-sensitivity to positive and negative effectors. FIG. 5E: In control mitochondria, the differences in Ca⁺⁺ required for mtPTP activation (ATR, P<0.01; ADP, P<0.01; t-H₂O₂, P<0.01; and DIA, P<0.001). FIG. 5F: In ANT-deficient mitochondria, the differences in Ca⁺⁺ required for mtPTP activation (t-H₂O₂, P<0.01; DIA, P<0.05; ATR and ADP had no effect).

FIGS. 6A and 6B show ANT1 and ANT2 expression and respiration of control and ANT-deficient liver mitochondria. Liver mitochondria were isolated from Ant1^{-/-}, Ant2^{fl/Y} (ANT-control) or Ant1^{-/-}, Ant2^{fl/Y}, Alb-cre (ANT-deficient) mice. FIG. 6A: Western blot analysis using antibodies against ANT1 (left) and ANT2 (right). Heart mitochondria were used as a positive control for the presence of ANT1. ANT1 and ANT2, 30 kDa. FIG. 6B: Oxygen consumption was measured using succinate as substrate in ANT-control (left) and ANT-deficient (right) mitochondria. MITOS, addition of 600 μg of mitochondrial protein. ADP, 125 nmol ADP; FCCP(uncoupler), 75 nM.

FIG. 7 examines oxidative phosphorylation enzyme and uncoupler protein analysis of ANT-deficient liver mitochondria. Mouse genotypes are as in FIG. 6. FIG. 7A: oxidative phosphorylation enzyme activities were measured in ANT-control (□) or ANT-deficient (▪) liver mitochondria isolated from three mice each. Respiratory complexes are designated by their number (I-IV). Fum, fumarase. The error bars represent the standard error of the means. FIG. 7B: Western blot analysis performed on mitochondrial protein from three control and three mutant mice. COI, 56 kDa, UCP2, 33 kDa, cytochrome c,11 kDa. FIG. 7C: Ponceau S stained blot from (b) illustrating the approximate equal protein loading.

FIGS. 8A-8B show liver mitochondrial mtPTP activation by ΔP depolarization, as monitored by mitochondrial swelling. Mouse genotypes are as in FIG. 6. FIG. 8A: ANT-control and FIG. 8B: ANT-deficient mtPTP activation monitored by light scattering. RR, ruthenium red, FCCP (1 μM). Traces with and without cyclosporin A, labelled.

FIG. 9 shows the analysis of FasL-induced apoptosis in ANT-deficient mouse hepatocytes. Primary hepatocytes from ANT-deficient and ANT-control mice were cultured on collagen-coated cover glasses and treated with Fas ligand and Actinomycin D. After 12 h, cells were stained with Hoechst 33258 and images were acquired with a Zeiss Axio-10 fluorescence microscope. Cells with chromatin condensation and nuclei fragmentation were counted as apoptotic.

FIG. 10 illustrates sFAS-ligand, TNFa, and Actinomycin D-induced apoptosis in ANT-deficient and ANT-control hepatocytes, apoptotic nuclei quantified (FIG. 9) after 12 h. Data presented are the average of three separate experiments.

FIG. 11 shows calcium ionophore-induced cell death in ANT-deficient and ANT-control mouse hepatocytes. Cells were treated with indicated concentrations of Br-A23187 for 1 h. Viability was measured by LDH release assay 1 h thereafter.

FIG. 12 shows the effects of cyclosporin A and z-VAD on viability and Br-A23187-induced cell death, as measured by LDH release. Cells were pretreated with either 1 μM cyclosporin A or 50 μM z-VAD for 30 min before addition of Br-A23187.

### DETAILED DESCRIPTION OF THE INVENTION

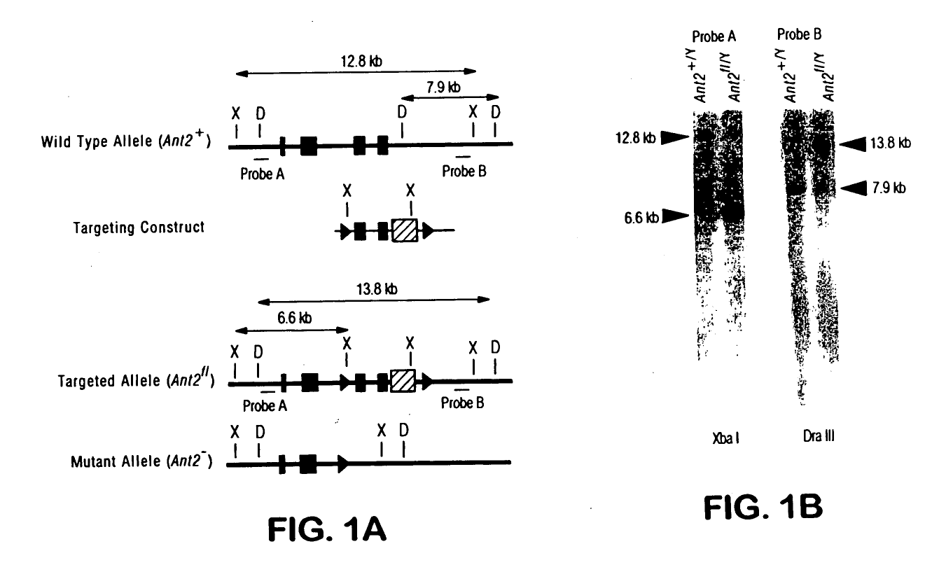

Mice deficient in Ant1 have been described previously (25). Mice deficient in Ant2 were prepared from embryonic stem cells in which loxP sites were inserted flanking exons 3 and 4 of Ant2 (FIG. 1). This conditional mutant allele of the X-linked Ant2 (Ant2^{fl}) could be combined with a tissue-specific cre recombinase transgene to remove exons 3 and 4 by site-specific recombination, thus generating the null allele (Ant2⁻).

Inactivation of the Ant2 gene in all tissues (global knockout) was achieved using a Cre recombinase transcribed from the mouse protamine-1 gene promoter (Prm-Cre). The protamine-1 promoter mediates expression of Cre during spermiogenesis. Therefore, Ant2^{fl/Y}, Prm-Cre males will transmit the null allele (Ant2⁻) to all females.

Females homozygous for the Ant2^{fl} allele (Ant2^{fl/fl}) were mated with Ant2^{fl/Y}, Prm-Cre males to generate females heterozygous for the Ant2^{fl} and Ant2⁻ alleles (Ant2^{-/fl}). These Ant2^{-/fl} females were viable since the average litter size of these crosses was normal (10.69±0.73 versus 10.80±1.24, in controls) and the observed genotypic frequencies of the progeny in these crosses were not significantly different from those predicted by Mendelian principles.

To generate Ant2-deficient mice we next crossed the Ant2^{-/fl} females with Ant2^{fl/Y}, Prm-Cre males. This would be expected to generate 50% Ant2-null progeny (Ant2^{-/Y} males or Ant2^{-/-} females). However, rather than giving viable progeny as seen for the Ant1^{-/-} mice 25, the average litter size at birth was reduced approximately 50% (4.65±0.38), with only about 5% of the Ant2-null animals being born. Moreover, Ant2-null animals that were live-born died by postnatal day 16.

To determine the reason for the embryonic lethality of the Ant2^{-/Y} and Ant2^{-/-} embryos, we set up timed matings between Ant2^{fl/Y}, Prm-Cre males and Ant2^{-/fl} females. Up to embryonic day 10.5 (e10.5) approximately equal numbers of mutant and control embryos were observed in each litter (4.5±0.8 mutants versus 3.3±0.3 controls (n=6 litters)). However, by e12.5 the numbers of mutant embryos were significantly reduced (2.2±0.1 mutants and 5.0±0.9 controls, P<0.05; (n—6 litters)), and by e14.5 living mutant embryos were rare (0.8±0.5 mutants and 4.3±0.6 controls, P<0.01; (n=4 litters)). Beyond e14.5, no viable Ant2-null embryos were recovered from timed matings (n=3 litters).

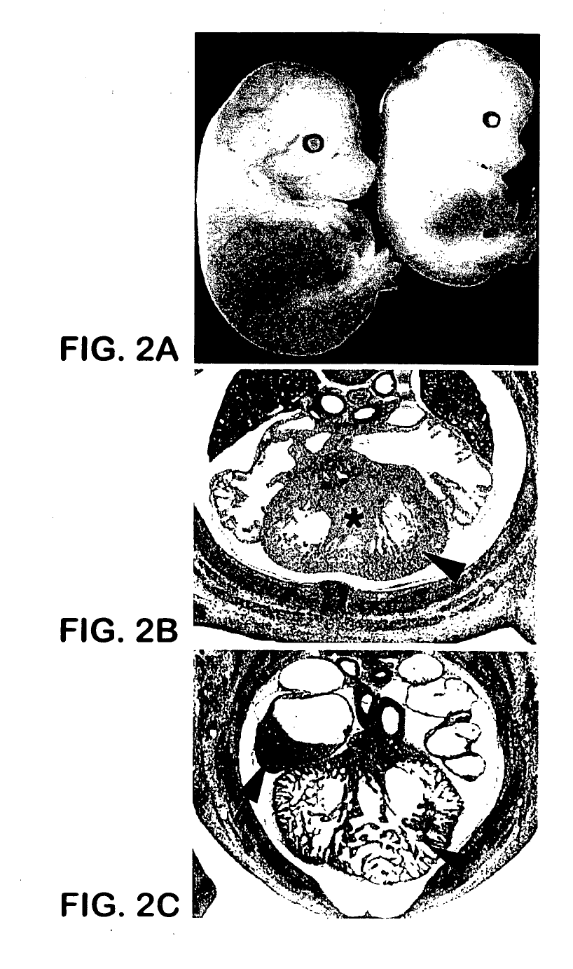

To identify the embryonic defect responsible for the fetal loss, control and mutant embryos were examined between e10.5 and e14.5. At e10.5, Ant2 null embryos had no overt abnormalities. However, by e12.5 the null embryos appeared pale and anemic and in many cases the pericardial sac appeared dilated and the yolk sac lacked blood. By e14.5 mutant embryos were developmentally retarded, with little evidence of blood in the peripheral vasculature (FIG. 2A). These observations suggest a defect in cardiac development, since a critical period of ventricle maturation occurs between e12.5 and e14.5 (26,27).

Cardiac defects were found in Ant2-null embryos (FIGS. 2A-2C). The histology of mutant and control embryos appeared normal at e10.5. However, by e12.5 the hearts in Ant2-null embryos displayed subtle but consistent abnormalities in formation of the interventricular septum. By e14.5 the mutant embryos exhibited pronounced cardiac abnormalities including malformed or absent interventricular septum, dramatically thinned ventricle walls, abnormally thinned trabeculae, and dilated atria (FIGS. 2B-2C). No other tissue in Ant2-null embryos showed gross pathological changes, although some tissues appeared underdeveloped.

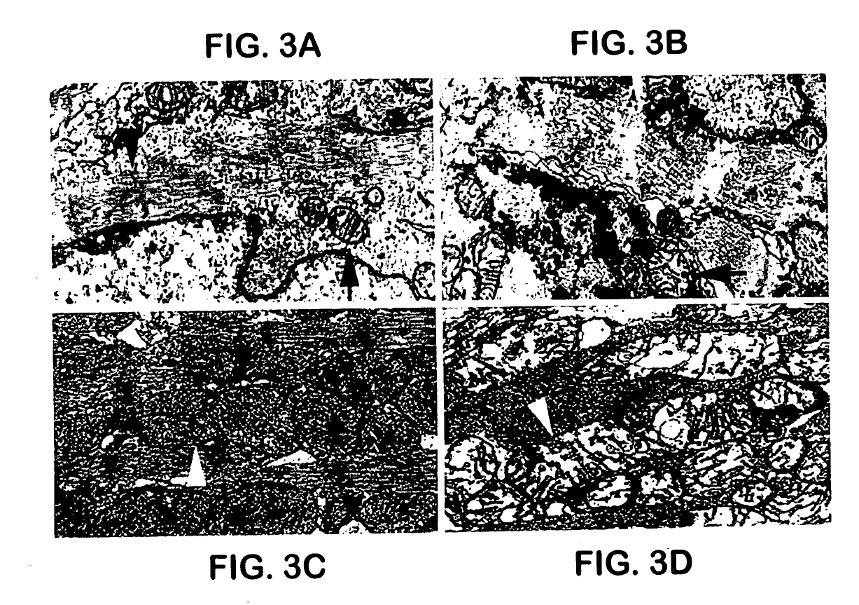

Ultrastructure analysis of Ant2-null hearts from e14.5 embryos revealed that the ventricular wall muscle fibers were disrupted and surviving myocardial cells contained large numbers of cytosolic electron-dense particles as well as unusual mitochondria containing inclusions (FIG. 3A, 3B). Mitochondrial abnormalities were even more pronounced in the cardiomyocytes of the Ant2-null animals that survived to term. The mutant heart mitochondria were large and swollen, with reduced cristae and in some cases intra-mitochondrial inclusions (FIG. 3C, 3D).

The embryonic cardiac defects of the Ant2-deficient mice were in stark contrast to the normal embryonic development of Ant1 null mice (25). One possible explanation for this striking difference could have been that only Ant2 is expressed in the embryonic heart, even though both Ant1 and Ant2 are expressed in the adult heart. To determine the embryonic expression patterns of Ant1 and Ant2 during cardiac development, mRNA levels were analyzed by in situ hybridization and real time PCR.

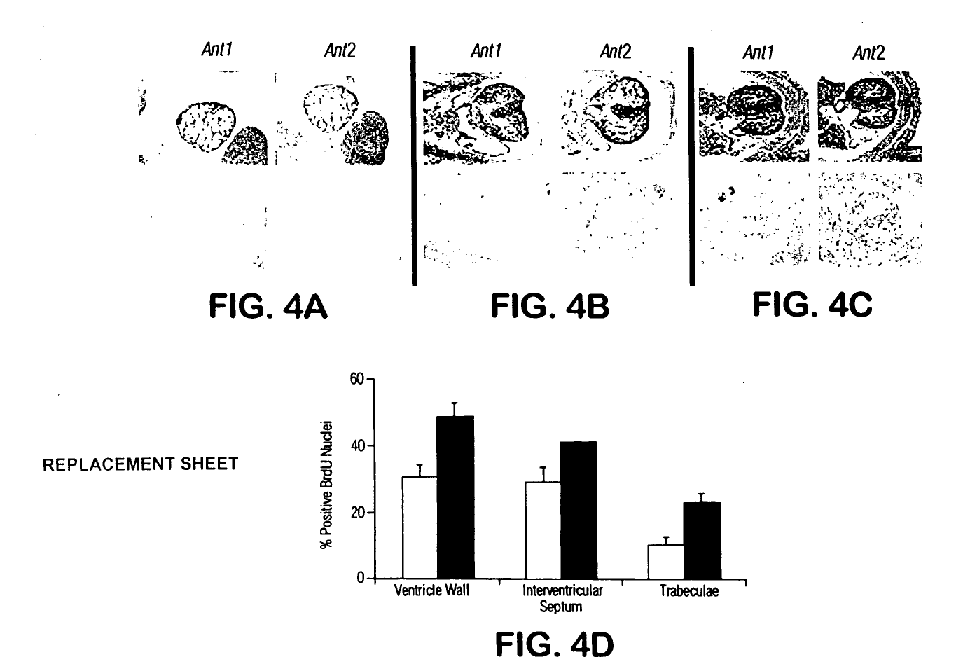

Analysis by in situ hybridization indicated both Ant1 and Ant2 mRNAs were expressed in the hearts of e10.5, e12.5, and e14.5 mouse embryos (FIGS. 4A-4C). Consistent with this finding, quantification of levels of Ant1 and Ant2 mRNAs by real time PCR indicated both Ants were expressed during cardiac development with the Ant1 mRNA being approximately five-fold more abundant than the Ant2 mRNA in e14.5 control hearts (3.96±1.12 fg/μl versus 0.69±0.11 fg/μl, respectively). Hence, Ant2-deficiency did not result in an absence of ANT in the cardiomyocytes.

To determine if the Ant2-deficiency caused the cardiac defect by limiting cardiomyocyte proliferation, embryonic cardiomyocyte DNA synthesis was monitored by BrdU incorporation (FIG. 4D). Surprisingly, the cardiomyocytes of the Ant2 null hearts contained a significantly higher proportion of replicating cells in the ventricle wall, interventricular septum, and trabeculae than did the hearts of control embryos. Thus, the cardiac defect of the ANT2-deficient embryos is not simply due to inhibition of cardiomyocyte proliferation.

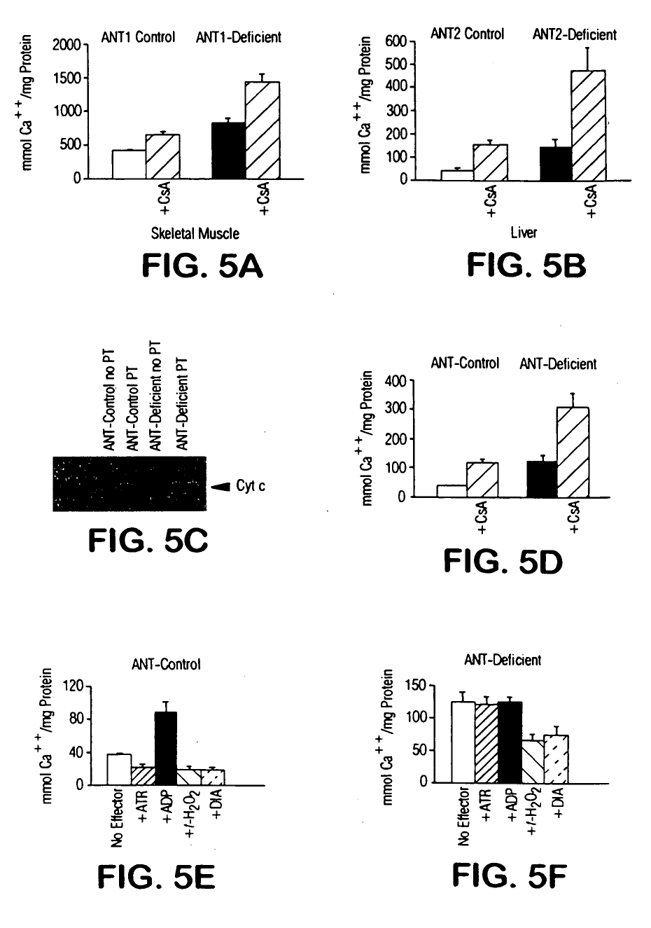

To determine if ANT1 or ANT2 deficiency affected mtPTP function, we analyzed the liver mitochondria of the 5% surviving neonatal Ant2-null mice and the skeletal muscle mitochondria of the Ant1-null mice. Since ANT2 is essentially the only ANT expressed in mouse liver and ANT1 is virtually the only ANT expressed in mouse skeletal muscle (9), the use of these tissues minimized possible effects on mtPTP activation by the presence of the other ANT isoform (FIGS. 5A, 5B).

The tendency of mitochondria containing either ANT1 or ANT2 to undergo the permeability transition was quantified by determining the amount of Ca⁺⁺ necessary to activate cyclophilin D and open the mtPTP. Opening of the pore was detected by the collapse of the mitochondrial membrane potential (ΔP).

The mitochondria were energized with succinate in the presence of TPP⁺. TPP⁺ is accumulated into the mitochondrial matrix in proportion to ΔP, and its concentration in the suspension medium is monitored using a TPP⁺ sensitive electrode. Aliquots of Ca⁺⁺ are then added to the reaction solution, with each addition causing a transient release of TPP⁺ as the Ca⁺⁺ is taken into the mitochondria at the expense of ΔP. The Ca⁺⁺ additions are repeated until sufficient Ca⁺⁺ has accumulated in the mitochondrial matrix to activate cyclophilin D and hence the mtPTP. At this juncture, ΔP collapses and the mitochondrial TPP⁺ is irreversibly released into the suspension medium. The amount of Ca⁺⁺ required to cause irreversible release of TPP⁺ provides a quantitative measurement of mtPTP sensitivity. Confirmation that the collapse of ΔP was due to the mtPTP was obtained by inhibiting cyclophilin D with cyclosporin A (CsA), thus increasing the level of Ca⁺⁺ necessary to open the pore and release the TPP⁺.

In skeletal muscle mitochondria, which contain predominantly ANT1, the inactivation of the Ant1 gene increased the Ca⁺⁺ required to activate the mtPTP by 99% (420±21.5 versus 837.5±42.7 nmol Ca⁺⁺/mg mitochondrial protein, P<0.0001) (FIG. 5A). Similarly, in liver mitochondria, which contain predominantly ANT2, the inactivation of the Ant2 gene increased the Ca⁺⁺ required to activate the mtPTP 59% (60.2±0.15 versus 95.7±5.97 nmol Ca⁺⁺/mg mitochondrial protein, P<0.05) (FIG. 5B). Thus, loss of either ANT1 or ANT2 blunts the mtPTP's sensitivity to Ca⁺⁺, confirming that both ANT2 and ANT1 are associated with the mtPTP. In mitochondria lacking both isoforms of ANT, three times more Ca⁺⁺ was required to activate the mtPTP than in those expressing ANT2 but not ANT1.

Surprisingly, however, the skeletal muscle mitochondria containing ANT1 required almost seven times more Ca⁺⁺ to initiate permeability transition than did liver mitochondria containing predominantly ANT2 (FIGS. 5A, 5B), a result consistent with the observations of others (28). Assuming that liver and muscle mitochondria are otherwise similar with respect to the mtPTP, these results demonstrate that ANT1 and ANT2 are not the same, and that they impart different properties to the mtPTP.

While skeletal muscle and liver express predominantly ANT1 and ANT2, respectively, small amounts of the other isoform can be detected with ultrasensitive methods (9). To eliminate a possible effect of a small amount of ANT1 on mtPTP activation in ANT2-deficient liver, we bred the Ant2^{fl} allele on to an Ant1^{-/-} background. We then inactivated Ant2 by the liver-specific expression of the cre recombinase transcribed from an albumin gene promoter (Alb-Cre).

Ant2^{fl/fl}, Ant1^{-/-} female mice were crossed with Ant2^{+/Y}, Ant1^{-/-}, Alb-Cre males. Based on a previous report, we anticipated that the Alb-Cre transgene would excise greater than 99.0% of the Ant2 genes in the developing hepatocytes (29). Half of the male progeny from this cross were predicted to be Ant1^{-/-}, Ant2^{fl/Y}, Alb-Cre, and thus be deficient for both Ant1 and Ant2 genes in their liver mitochondria.

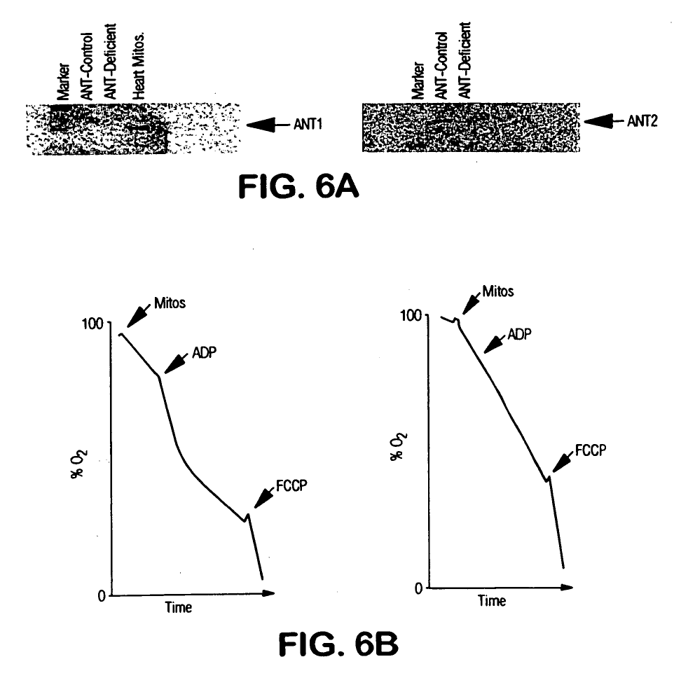

The absence of ANT1 and ANT2 protein in the Ant1^{-/-}, Ant2^{fl/Y}, Alb-Cre liver mitochondria was confirmed by western blot analysis using isoform-specific antibodies (FIG. 6A). Complete ANT deficiency was further confirmed by demonstrating that respiration in mitochondria from Ant₁^{-/-}, Ant2^{fl/Y}, Alb-Cre animals could not be stimulated by the addition of ADP (FIG. 6B). Hence, the liver mitochondria of the Ant1^{-/-}, Ant2^{fl/Y}, Alb-Cre mice were totally devoid of ANT.

While ADP did not stimulate respiration, consistent with the lack of ANT, we did observe that the endogenous respiration rates of the ANT1 and ANT2-deficient mitochondria were almost twice that of control mitochondria (34.58±1.6 nmol O/min/mg versus 18.12±1.1 nmol O/min/mg) (FIG. 6B). Moreover, the mitochondrial membrane potential of the ANT-deficient mitochondria was significantly higher than that of controls (191.7±4.9 mV versus 172.9±3.5 mV).

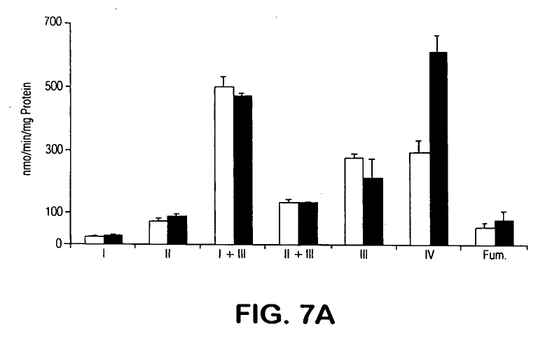

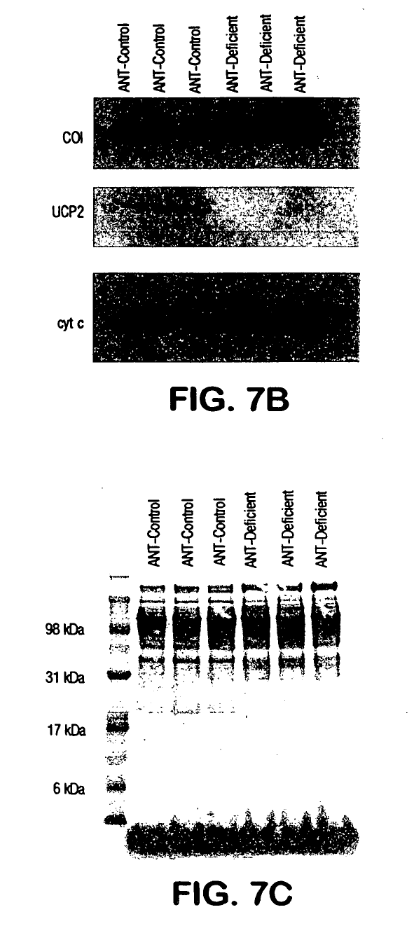

To determine the molecular basis of the increased respiration rate and membrane potential, we examined the levels of the mitochondrial respiratory chain enzymes and uncoupler protein (FIG. 7). Analysis of the specific activities of the oxidative phosphorylation enzyme complexes of the ANT-deficient mitochondria revealed that complex IV (COX) was increased more than two fold over that of the controls (P<0.01) (FIG. 7A). This was confirmed by western blot analysis which revealed that the mitochondrial cytochrome c oxidase subunit In (COI) and cytochrome c proteins were more abundant in the ANT-deficient mitochondria (FIG. 7B). Thus, the increased respiration rates are likely to be the result of the specific upregulation in COX activity, perhaps as a compensation for the defect in mitochondrial energy production. If so, COX activity must be a major modulator of the electron transport chain.

To determine the reason for the increased mitochondrial membrane potential, we analyzed the expression of the systemic uncoupler protein, UCP2. UCP2 was found to be down-regulated to undetectable levels in the ANT-deficient mitochondria, as shown by western blot analysis (FIG. 7B). This would be a reasonable way to compensate for reduced mitochondrial ATP. Since the nucleus would need to increase ΔP to stimulate the ATP synthase, it could reduce proton leak by shutting off UCP2.

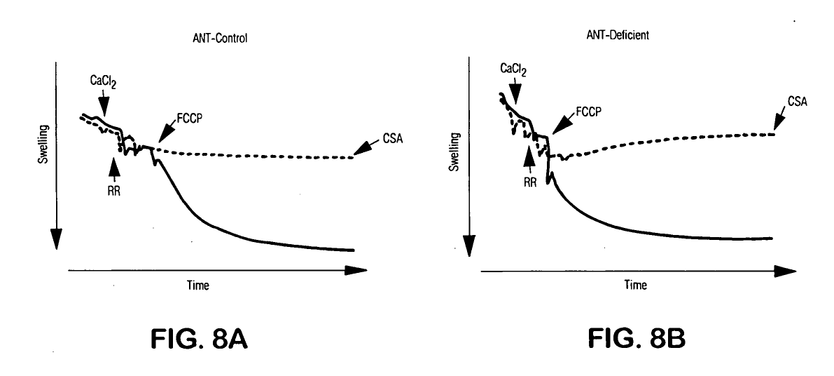

Having confirmed that the liver mitochondria of Ant1^{-/-}, Ant2^{fl/Y}, Alb-Cre mice lacked both ANT1 and ANT2, we used these mitochondria to continue our investigation of the role of ANT in the structure and regulation of the mtPTP. To determine if the mtPTP could still exist in the absence of ANT, we isolated mitochondria from livers of Ant1^{-/-}, Ant2^{fl/Y}, Alb-Cre (ANT-deficient) and Ant1^{-/-}, Ant2^{fl/Y} (ANT-control) mice. Taking advantage of the fact that the mtPTP is a voltage-dependent channel, we determined if the mtPTP could still be activated by the uncoupler FCCP in the presence of Ca⁺⁺³⁰ (FIGS. 8A-8B). The opening of the pore was monitored by following mitochondrial swelling through decreased light scattering. Surprisingly, ANT-deficient and ANT-control mitochondria responded equally well to FCCP-induced mtPTP activation, and both responses were comparably inhibited by CsA (FIGS. 8A-8B). CsA completely inhibited the swelling of both the ANT-control and ANT-deficient mitochondria. Thus, the mtPTP is fully functional in the absence of ANT, demonstrating that the ANTs are not essential components of the mtPTP ion channel.

To further confirm the integrity of the mtPTP in the ANT-deficient mitochondria, we investigated cytochrome c release following Ca⁺⁺-induced permeability transition (FIG. 5). Ca⁺⁺ was added to TPP⁺-loaded mitochondria, and once the TPP⁺ was released, the mitochondria were removed by centrifugation and the supernatants analyzed by western blot for soluble cytochrome c (FIG. 5C). Cytochrome c was found to be released from both the ANT-deficient and ANT-control mitochondria. Moreover, since the ANT-deficient mitochondria contained more cytochrome c than controls, the mutant mitochondria released more cytochrome c. Hence, ANTs are not required for mtPTP-induced cytochrome c release.

While the ANTs are not essential components of the mtPTP, their absence did reduce the sensitivity of the mtPTP to Ca⁺⁺ activation in the ANT1-deficient muscle and ANT2-deficient liver mitochondria. This was confirmed in the liver mitochondria of the ANT1 and 2-deficient animals (Ant1^{-/-}, Ant2 μl, Alb-Cre) which required three times more Ca⁺⁺ to activate the mtPTP than did mitochondria isolated from controls (Ant₁^{-/-}, Ant2^{fl/Y}) (FIG. 5D). This demonstrates that the ANTs play a role in regulating the mtPTP.

To characterize the regulatory role of the ANTs further, we took advantage of the known modulation of the mtPTP by specific positive and negative effectors. Tert-butyl hydroperoxide (t-H₂O₂) and diamide (DIA) are both inducers of the mtPTP, t-H₂O₂ being a non-specific oxidant and DIA being a specific —SH group oxidant. Both of these compounds activated the mtPTP, whether or not ANT was present (FIGS. 5E, 5F). Atractyloside (ATR) and ADP are positive and negative effectors of the mtPTP that are thought to act by binding to the cytosolic face of the ANT. While ATR activated the mtPTP in ANT-control mitochondria, it had no effect on ANT-deficient mitochondria. Similarly, while ADP strongly inhibited the activation of the mtPTP in ANT-control mitochondria, it was without effect on the ANT-deficient mitochondria (FIGS. 5E, 5F). Thus, the absence of ANT results in the loss of the modulation of the mtPTP by ANT ligands including adenine nucleotides.

Current models have envisioned that the ANT constitutes a core element of the mtPTP (14,23), but our results using ANT1 and ANT2-deficient mitochondria have revealed that the ANTs are not an essential component of the mtPTP inner membrane ion channel. Rather the ANTs are required for the sensitization of the mtPTP to Ca⁺⁺ activation and the modulation of the pore by specific ligands of the ANTs. Therefore, the ANTs could regulate the mtPTP as either an integral or peripheral component of the mtPTP, a conclusion consistent with earlier visions of the mtPTP structure (31).

One of the unexpected discoveries from this study was that ANT1 and ANT2 deficiency have different effects. Ant1-deficient mice progress through cardiac development normally, while Ant2-deficient mice died during gestation due to cardiac abnormalities. Yet both ANT isoforms are expressed in the heart during gestation. As young adults, Ant1-deficient mice were found to develop mitochondrial myopathy and multiple mtDNA deletions (25), a result which foreshadowed the discovery that ANT1 missense mutations in humans can cause autosomal dominant progressive external ophthalmoplegia with multiple mtDNA deletion syndrome (32). Without wishing to be bound by any particular theory, it is believed that certain forms of human congenital cardiomyopathy are linked to mutations in the human ANT2 gene as well.

The reason for the difference in the cardiac development of the ANT1 and ANT2-deficient mice is currently unclear. It is unlikely that the cardiac defect is due to an ATP deficiency, since Ant1 mRNA is present in the embryonic hearts at five times the level of Ant2 mRNA, and ANT1 must be efficient at ATP transport since it is expressed in those tissues that require high mitochondrial ATP output including heart, muscle, and brain. Also, the cardiomyocytes of the ANT2-deficient mice were found to replicate faster than in ANT2-containing cardiomyocytes, which would belie ATP limitation.

Another possibility is that the increased proliferation of the ANT2-deficient cardiomyocytes compromised normal differentiation. The heart, muscle, and brain mitochondria of Ant1^{-/-} animals produce increased amounts of reactive oxygen species, specifically hydrogen peroxide (25,33), and increased production of hydrogen peroxide has been shown to be a potent nuclear mitogen (34,35). Hence, ANT2-deficiency in the heart might also increase hydrogen peroxide production, which could drive cardiomyocyte replication and block differentiation. However, if this were the mechanism, there would have been a similar cardiac developmental defect in Ant1^{-/-} mice which are known to produce excessive hydrogen peroxide (25,33). There was not. Hence, uncontrolled proliferation is an unlikely explanation.

A third possibility is that Ant2-deficiency inhibited apoptosis, thus blocking cardiac remodeling. A decrease in programmed cell death could be perceived as an increase in the number of proliferating cells. Support for this hypothesis comes from the demonstration that mice defective in the genes for the apoptosis proteins caspase 8 or FADD/MORT1 develop ventricular defects between e10.5 and e14.5 similar to those seen in our Ant2-deficient mice (36,37). If Ca⁺⁺ activation of the mtPTP is important for inducing apoptosis in developing cardiomyocytes, then the much higher resistance of mtPTPs associated with ANT1, than mtPTPs associated with ANT2, to activation by Ca⁺⁺ could have reduced cardiomyocyte apoptosis in the ANT2-deficient embryonic hearts. A greater role for ANT2 in modulating the mtPTP relative to ANT1 is also consistent with the observation that ANT2 interacts with VDAC at all times, but ANT1 only interacts with VDAC in the presence of ATR (38). Thus, apoptosis might be inhibited in the hearts that lacked ANT2, but not in those that lacked ANT1.

Whatever the biochemical basis for the difference in phenotypes between the Ant1^{-/-} and Ant2^{-/Y} mice, it is clear that the ANTs are not responsible for the formation of the mtPTP ion channel that spans the mitochondrial inner membrane. However, the ANTs play a vital role in the modulation of the mtPTP in relation to vital physiological signals such as Ca⁺⁺ and adenine nucleotides, linking energy metabolism and programmed cell death.

The capacity of the mtPTP in the ANT-deficient mitochondria to initiate apoptosis was also confirmed by demonstrating that cytochrome c could still be released from the ANT-deficient mitochondria following Ca⁺⁺-induced permeability transition. Ca⁺⁺ was added to TPP⁺-loaded mitochondria, until the TPP⁺ was irreversibly released. Then the mitochondria were removed by centrifugation and the supernatants were analyzed by western blot for soluble cytochrome c. Cytochrome c was found to be released from both the ANT-deficient and ANT-control mitochondria, while the inner membrane COI protein remained associated with the mitochondrial pellets.

To determine if the ANTs provide a regulatory function for the mtPTP, ANT-deficient and control liver mitochondria were loaded with TPP⁺ and then Ca⁺⁺ was added in the presence or absence of various mtPTP effectors until the TPP⁺ was irreversibly released. The relative levels of Ca⁺⁺ required for permeability transition were then compared. Tert-butyl hydroperoxide (t-H₂O₂) and diamide (DIA) are both inducers of the mtPTP, t-H₂O₂ being a non-specific oxidant and DIA being a specific —SH group oxidant. Both of these compounds activated the mtPTP, whether or not ANT was present. Atractyloside (ATR) and ADP are positive and negative effectors of the mtPTP, respectively, that are thought to act by binding to the cytosolic face of the ANT. While ATR activated the mtPTP in ANT-control mitochondria, it had no effect on ANT-deficient mitochondria. Similarly, while ADP strongly inhibited the activation of the mtPTP in ANT-control mitochondria, it was without effect on the ANT-deficient mitochondria. Thus, the absence of ANT results in the loss of the modulation of the mtPTP by ANT ligands including adenine nucleotides.

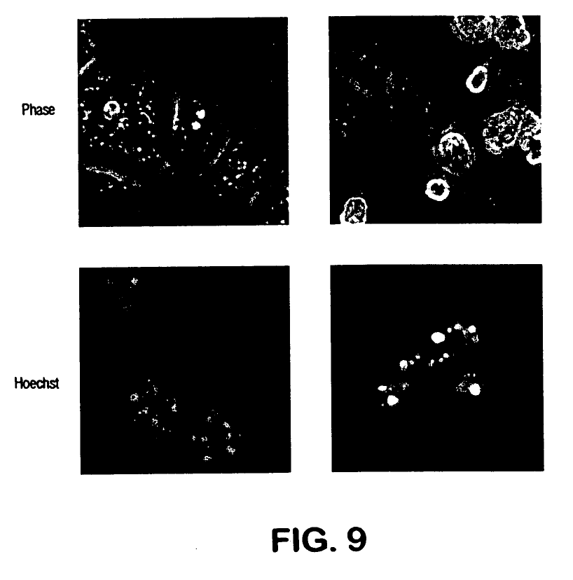

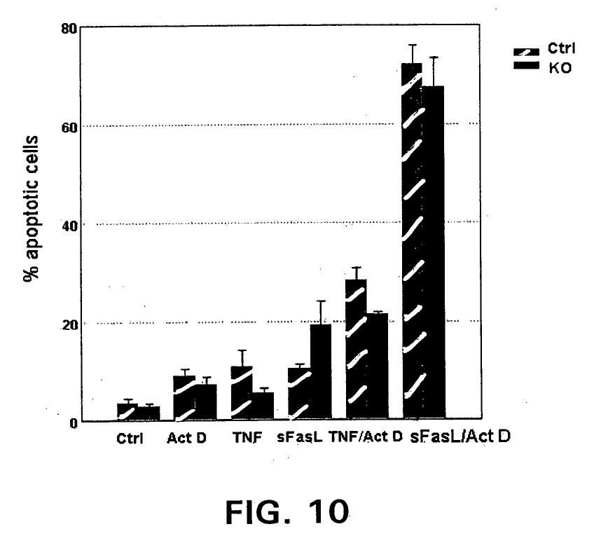

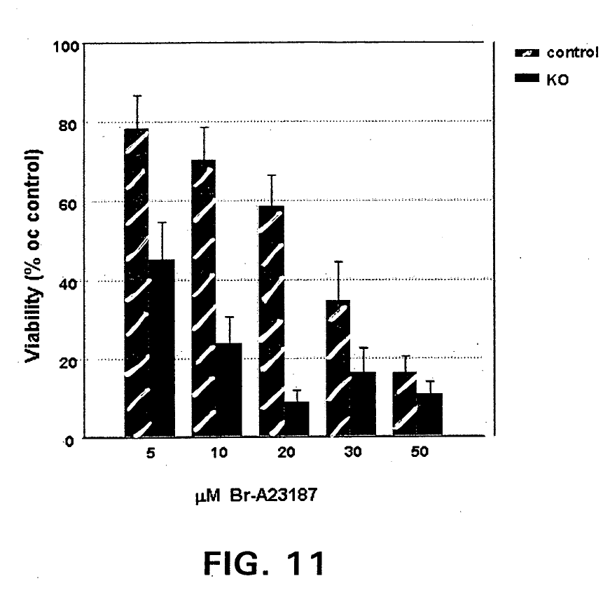

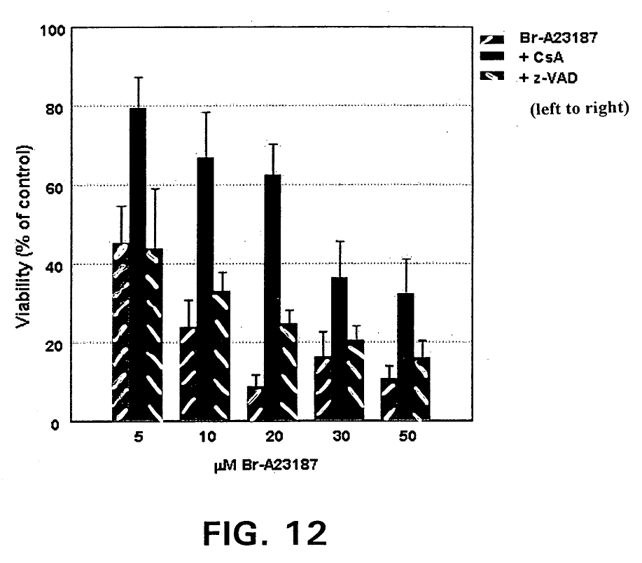

MtPTP has previously been shown to be involved in both receptor-mediated apoptosis (51) and calcium-mediated cell death (50). To determine the effect of ANT deficiency on the induction of cell death, mouse hepatocytes were isolated from ANT-deficient (Ant1^{-/-}, Ant2^{fl/y}, Alb-Cre) and ANT-control (Ant1^{-/-}, Ant2^{fl/Y}) mice and exposed to inducers of apoptosis and necrosis. Treatment with Fas ligand+Actinomycin D (51) for 12 hours killed about 70% of both ANT-deficient and ANT-control hepatocytes as detected by nuclear apoptosis using Hoechst 33258 staining (FIGS. 9 and 10). TNFα+Actinomycin D killed 20-25% of both ANT-deficient and ANT-control cells whereas TNFa, or Actinomycin D alone did not induce apoptosis (FIG. 10). Treatment of ANT-deficient and ANT-control hepatocytes with the calcium ionophore, Br-A23187, resulted in extensive loss of cell viability in both strains, monitored by lactate dehydrogenase release (FIG. 11). The ANT-deficient cells were more sensitive to Br-A23187 than were the ANT-control cells (5 μM Br-A23187 killing 20% of ANT-control and 55% of ANT-deficient cells with 50 μM killing 80-90% of both cell types). The increased sensitivity of the ANT-deficient hepatocytes to the calcium ionophore may indicate that ANT-deficiency pre-sensitizes the mitochondria to induction of the mtPTP. Br-A23187 also induced mitochondrial swelling in the ANT-deficient cell in vivo. The cell death in the ANT-deficient hepatocytes induced by Br-A23187 was strongly inhibited by cyclosporin A but not by the general caspase inhibitor z-VAD (FIG. 12). Hence, the hepatocyte toxicity observed is dependent on the mtPTP, and therefore, ANT is not required for mtPTP activation or mitochondria-mediated cell death.

Mice of the genotype nestin-cre, Ant2^{fl/y} were also generated; these mice appeared normal in gross appearance. Behavior, reproduction, size and eating patterns were all normal, throughout the 10 month observation period. The brains of these mice exhibited normal morphology upon dissection. The nestin transcription regulatory sequences and their use in expression of heterologous sequences are described in WO 01/36482, published May 25, 2001. These sequences direct the expression of an associated coding sequence in multipotent, especially in neural stem and progenitor cells as well as in cells of the developing liver, tooth, heart, pancreas, intestinal tract and retina.

Mice of the genotype Ant1^{-/-}, nestin-cre, Ant2^{fl/y} were dead at birth. They are found in the fetal position, resembling that held during embryogenesis in utero. Without wishing to be bound by theory, it is believed that mouse embryos which lack Ant2 in the developing nervous system sustain severe neurological defects. Certain human stillbirths have this appearance.

Cell-specific (or tissue-specific expression of a structural gene (in the present context the cre recombinase coding sequence) is controlled by cell-specific regulatory (or control) sequences positioned such that the structural gene is under their regulatory control. The term cell-specific regulatory sequence refers to in DNA sequences or sequence elements required for cell-specific expression of a coding sequence, including cell-specific enhancers, cell-specific upstream regulatory sequences and cell-specific promoters, as well as those non-cell-specific transcription control sequences including non-cell-specific promoters and polyadenylation signals necessary for expression of a coding sequence in a eukaryotic cell. Maximum selective control of a heterologous structural gene is likely to be obtained when the heterologous gene is placed in a position relative to the regulatory sequences that is similar to the positioning of the homologous gene normally controlled by those sequences. Cell-specific enhancers as well as cell-specific promoters have been identified. In some cases, both a cell-specific promoter and enhancer will function, as, for example, in selective expression of immunoglobulin genes. Selective expression of the heterologous gene can be effected by, for example, trans-activation of cell-specific regulatory sequences.

Selectivity of expression of a heterologous structural gene by the regulatory sequences of another gene is achieved by replacing the coding region normally controlled by those sequences with the heterologous structural gene retaining the full complement of regulatory sequences (including a promoter and any enhancer or upstream regulatory sequences). Selective expression can also be achieved with chimeric regulatory sequences in which regulatory units (i.e., enhancers, promoters, etc.) are combined to obtain selective expression. It is not necessary, however, for use in the present invention, that the specific regulatory sequences which control cell-selective or tissue-specific expression be characterized. It is only necessary that the DNA sequences sufficient for efficient and selective control be localized to within a reasonably small, about 1-2 kb, region of DNA, such that the sequences are useful for the construction of chimeric genes. It is often the case that upstream cell-selective regulatory sequences are located within about 1-2 kb upstream of the initiation site of genes that are expressed in a cell-selective manner.

A number of genes contain regulatory sequences which selectively activate expression of the gene coding sequence which is under their regulatory control in only certain cell (or tissue) types. Such genes are designated as cell-specific and the regulatory sequences that mediate such expression are designated cell-specific regulatory sequences or sequence elements. Expression from cell-specific regulatory sequences can be controlled in a variety of ways. For example, trans-acting factors can stimulate expression by binding to the enhancer or to sequences in or near the promoter with or without a cofactor. Additionally, expression can be stimulated by factors that render transcription repressors incapable of binding to the cis repressor-responsive element or otherwise nonfunctional.

The degree of selectivity of expression can vary significantly from very stringent to merely preferential. With very stringent control, gene expression is effectively exclusive to a single cell or tissue type with non-detectable levels of expression in other cells. In contrast, a preferentially expressed gene may be detected in a variety of cell types; however, the level of gene expressed may be higher in one or several cell types. Intermediate levels of selectivity will display intermediate levels of stringency of gene expression with variations in the basal levels of gene expression in non-target cells or in the range of cell types in which the gene is expressed. For use in the DNA constructs and methods of the present invention, it is preferred that the cell-selective regulatory sequences be sufficiently restrictive such that the level of expression of the Cre recombinase in target cells is sufficient to delete the loxP-flanked Ant2 sequences in that tissue, while the level of expression in non-target cells is not sufficient to result in functional inactivation of the Ant2 gene in those non-target cells or tissues. The regulatory control sequences of elastase and gamma crystallin are sufficiently stringent to be operable in the present invention. The high stringency of control of elastase and crystallin gene regulatory sequences has been demonstrated by the selective expression of oncogenes in transgenic mice (See, e.g., Ornitz et al. (1985) Cold Spring Harbor Symp. Quant. Biol. 50:389-409). In contrast, the regulatory sequences which control expression of metallothionein genes are not, themselves, sufficiently selective for use in combination with the cre recombinase coding sequence. The cell selectivity of control conferred by the regulatory sequences of immunoglobulin genes, both Ig H enhancer and immunoglobulin promoter, is sufficiently stringent for selective elimination of Ant2 activity in B-cells. In principle, any cell-specific regulatory sequences which display expression stringency equivalent to those of the albumin, elastase, crystallin, or immunoglobulin genes can be employed in the present invention.

Monoclonal or polyclonal antibodies, preferably monoclonal, specifically reacting with a protein of interest may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York. Abbreviations and nomenclature, where employed, are deemed standard in the field and commonly used in professional journals such as those cited herein.

All references cited in the present application are incorporated by reference herein to the extent that they are not inconsistent with the present disclosure.

The following examples are provided for illustrative purposes, and are not intended to limit the scope of the invention as claimed herein. Any variations in the exemplified articles which occur to the skilled artisan are intended to fall within the scope of the present invention.

### EXAMPLES

### Example 1

### Construction of the Ant2 and/or Ant1 Knockout Animals and Genotyping

All mice were on a hybrid background consisting of a mixture of 129S4 and C57BL6 strains, fed Purina Labdiet 5021, and housed at 20° C. with a 13/11-hour light/dark cycle. Animals were killed by cervical dislocation. All procedures were performed in accordance with Emory University's ethical guidelines outlined in a protocol approved by the Institutional Animal Care and Use Committee.

Ant1 knockout mice were generated as described previously (25; 48).

Mice with a liver-specific conditional mutant allele of Ant2 were generated via gene-targeting in embryonic stem (ES) cells. A targeting vector with exons 3 and 4 flanked by loxP sites (Ant2^{fl}) and containing a PGK-neo cassette was electroporated into the male mouse ES cell line AK7.1. Cells were selected in G418 and clones with a proper targeting event screened by Southern blot using multiple probes (FIG. 1). Site-specific recombination between the loxP sites removed the last 1/3 of the ANT2 protein and the PGK-neo cassette, including putative transmembrane domains 5 and 6, and generated a non-functional protein. When this conditional mutant allele was transmitted through the germ line and combined with a Cre recombinase transgene transcribed through the liver-specific albumin promoter (Alb-Cre), exons 3 and 4 of the Ant2 gene were removed by site-specific recombination. Because the Alb-Cre transgene is known to excise more than 99% of the Ant2^{fl} genes in the developing hepatocytes (29), combining the Ant2^{fl} allele with the Alb-Cre transgene generates an Ant2-null liver (Ant2⁻).

When Ant2^{fl/fl} female mice were crossed with Ant2^{+/Y}, Alb-Cre hemizygous males, half of the male progeny generated were Ant2^{fl/Y}, Alb-Cre, and thus null for Ant2 in their liver. While liver expresses predominantly ANT2, small amounts of the ANT1 isoform can be detected with ultrasensitive methods⁹. To eliminate any possible effect of this small amount of ANT1 on mtPTP, we bred the Ant2^{fl} allele on to an Ant1^{-/-} background²⁵. Thus, we were able to generate mice which lacked ANT2 in their liver (Ant2^{fl/Y}, Alb-Cre) with or without a systemic Ant1-deficiency. Since analyses of Ant1 and Ant2 double-knockout (Ant1^{-/-}, Ant2^{fl/Y}, Alb-Cre) mice provide the most rigorous test for the effect of the ANTs on mouse liver mtPTP function, these experiments will be emphasized here. However, parallel studies on Ant1^{+/+}, Ant2^{fl/Y}, Alb-Cre animals gave the same results.

The absence of ANT1 and ANT2 protein in the Ant1^{-/-}, Ant2^{fl/Y}, Alb-Cre liver mitochondria was confirmed by western blot analysis using isoform-specific antibodies (FIG. 2a). Complete ANT deficiency was further confirmed by demonstrating that respiration in mitochondria from Ant1^{-/-}, Ant2^{fl/Y}, Alb-Cre animals could not be stimulated by the addition of ADP (FIG. 2b). Hence, the liver mitochondria of the Ant1^{-/-}, Ant2^{fl/Y}, Alb-Cre mice were totally devoid of ANT.

Ant1 and Ant2 were genotyped by PCR, Ant1 as described (25) and Ant2 using the forward primer 5′ ACTCAACCTAGGGCCTTGTG 3′ (SEQ ID NO:1) and the reverse primer 5′ GGGAGCATTCCTGAAAAATAA 3′ (SEQ ID NO:2) (35 cycles of PCR: 94° C. for 20 secs, 56° C. for 30 secs, and 72° C. for 40 secs) to detect the targeted (485 bp) and wild type (384 bp) alleles. The mutant Ant2 allele (850 bp) was detected with the same forward primer and the reverse primer 5′ GACTTACCCTCCACGACAGC 3′ (SEQ ID NO:3) (35 cycles, 94° C. for 20 secs, 65° C. for 30 secs, and 72° C. for 60 secs).

### Example 2

### Histology, Ant Expression, and Cardiomyocyte Proliferation Analyses

For light microscopy, embryos were fixed overnight in 4% paraformaldehyde, and processed for hematoxylin and eosin staining. For transmission electron microscopy, cardiac tissue was fixed with 4% glutaraldehyde, stained, embedded, sectioned, and post-stained. Specimens were examined with a Philips CM-10 electron microscope.

In situ hybridization analyses (39) were performed using digoxygenin(DIG)-labeled riboprobes (40). The riboprobes encompassed cDNA nucleotides 817 to 1093 for Ant1⁴¹ and 834 to 1244 for Ant2 (41).

To assess cardiomyocyte proliferation, pregnant females were injected with 50 mg of BrdU/kg body weight two hours before sacrificing. The embryos were fixed overnight in 2% paraformaldehyde, processed in sequential sucrose concentrations of 10, 15, and 20%, incubated overnight at 4° C. in a solution of 1 part cryo-OCT and 1 part 20% sucrose, and embedded in a solution of 3 parts cryo-OCT and 1 part 20% sucrose. Specimens were cryo-sectioned at 6 microns, depurinated, neutralized, permeabilized, and incubated with goat anti-BrdU antibody (Harlan Sera-Lab, Leicestershire, England). A sheep anti-goat antibody (Jackson Labs, West Grove, Pa.) conjugated with fluorescein was used to detect BrdU-incorporation. Sections were counterstained using 5 uM Hoechst 33342. Fluorescein and Hoechst fluorescence was detected using a Zeiss-Axiophot™ microscope equipped with fluorescent optics. Three control and three mutant embryos were analyzed with approximately 500 heart nuclei counted per embryo.

### Example 3

### Western Blot Analysis

Isoform-specific ANT1 and ANT2 antibodies (25), and antibodies recognizing COI, UCP2, and cytochrome c (Molecular Probes, Santa Cruz, and Zymed) were reacted to isolated mitochondrial protein (20 μg) or supernatants separated by SDS-PAGE and blotted onto nitrocellulose.

### Example 4

### Tissue Harvesting and Mitochondrial Isolation

Tissues were harvested and the mitochondria isolated by liver homogenization or skeletal muscle slicing and homogenization, followed by differential centrifugation (42,43). The protein concentration was determined by using the Coomassie Stain kit (Pierce Chemical Co., Rockford, Ill.).

### Example 5

### Mitochondrial Respiration, Oxidative Phosphorylation Enzymology, and mtPTP

Respiration and oxidative phosphorylation enzyme activities were measured polarographically and spectrophotometrically (42). The membrane potential was calculated from the mitochondrial uptake of TPP⁺ using a TPP⁺ sensitive electrode (44-47). The mtPTP was examined in liver 10-week-old Ant1^{-/- or +/+}, Ant2^{fl/Y}, Alb-Cre +/−animals.

The calcium sensitivity of the mtPTP was calculated from 10 nmol CaCl₂ additions to a reaction vessel containing 1 mg of mitochondrial protein in 1.0 ml of 250 mM sucrose, 10 mM MOPS, 2 mM K₂HPO₄, pH 7.2, and 5 mM succinate. Irreversible release of TPP⁺ from the mitochondria was indicative of mtPTP activation. The following mtPTP effectors were employed: ATR, atractyloside, ADP, adenosine diphosphate, t-H₂O₂, tert-butyl hydrogen peroxide, DIA, diamide, CsA, cyclosporin A.

The mtPTP was examined in liver from neonatal Ant2-null animals, skeletal muscle from 3-month-old Ant1^{-/-} mice, and liver from 10-week-old Ant1^{-/-}, Ant2^{fl/Y}, Alb-Cre animals. The amount of Ca⁺⁺ required for mtPTP activation in liver mitochondria did not significantly differ between neonates and 3-month-old wild type mice (60.2±0.15 nmol Ca⁺⁺/mg protein versus 41.46±4.02 nmol Ca⁺⁺/min/mg protein, respectively), indicating that mtPTP results derived from tissues of these ages can be cross compared.

The voltage dependence of the mtPTP was assessed by depolarizing mitochondria with 1 uM FCCP (carbonyl cyanide p-(trifluoromethoxy)phenylhydrazone). Mitochondria (1 mg protein) were incubated in 1.5 ml of the above reaction medium with 16.5 nmol CaCl₂. After two minutes 0.1 uM ruthenium red was added followed by FCCP. Mitochondrial swelling indicating mtPTP activation was monitored by light scattering at 546 nm for 10 minutes, and confirmed by inhibition of swelling by 1 μM cyclosporin A.

### Example 6

### Induction of Cell Death in Primary Hepatocytes

Hepatocytes were isolated from 12 to 15 month old anesthetized mice, perfused in situ with collagenase-dispase medium (Invitrogen, Carlsbad, Calif.). The hepatocytes were gently released, filtered and cultured on collagen coated cover glasses or plates in Waymouth's MB-752/1 medium containing 27 mM NaHCO₃, 2 mM L-glutamine, 10% fetal calf serum, 0.5 □g/ml insulin, 0.5 □g/ml transferrin, 0.5 ng/ml sodium selenate and 100 nM dexamethasone (49).

Hepatocytes were treated with recombinant murine 100 ng/ml TNFα (R&D Systems, Minneapolis, Minn.) or 4 ng/ml of the human recombinant Fas ligand (Upstate, Lake Placid, N.Y.) with or without 0.2 μg/ml Actinomycin D (51). For calcium-induced cell death, cells were challenged with from 5 to 50 μM Br-A23187, without or with a 30 minute pretreatment of either 1 μM cyclosporin A or 50 μM z-VAD (50).

Fas ligand-TNFα+Actinomycin D induced apoptosis was monitored by detection of apoptotic nuclei by staining with 10 μg/ml of Hoechst 33258 (Molecular Probes, Eugene, Oreg.) (9). Br-A23187-induced cell death after 1 h treatment was monitored by measuring the percentage of lactate dehydrogenase (LDH) released into the culture medium as a percentage of the activity in a Triton X-100 total cell lysate.

### Example 7

### Statistical Analysis

Data analysis was carried out with GRAPHPAD PRISM software (GraphPad, San Diego, Calif.). P values represent the results of the Students unpaired t test.

### REFERENCES CITED IN THE TEXT OF THIS APPLICATION

1. Liu, X., Kim, C. N., Yang, J., Jemmerson, R. and Wang, X. Induction of apoptotic program in cell-free extracts: requirement for dATP and cytochrome c. Cell 86, 147-57. (1996).
2. Newmeyer, D. D., Farschon, D. M. and Reed, J. C. Cell-free apoptosis in Xenopus egg extracts: inhibition by Bcl-2 and requirement for an organelle fraction enriched in mitochondria. Cell 79, 353-64. (1994).
3. Riccio, P., Aquila, H. and Klingenberg, M. Purification of the carboxy-atractylate binding protein from mitochondria. FEBS Lett. 56, 133-8. (1975).
4. Nicolli, A., Basso, E., Petronilli, V., Wenger, R. M. and Bernardi, P. Interactions of cyclophilin with the mitochondrial inner membrane and regulation of the permeability transition pore, and cyclosporin A-sensitive channel. J. Biol. Chem. 271, 2185-92 (1996).
5. Halestrap, A. P., Woodfield, K. Y. and Connem, C. P. Oxidative stress, thiol reagents, and membrane potential modulate the mitochondrial permeability transition by affecting nucleotide binding to the adenine nucleotide translocase. J. Biol. Chem. 272, 3346-54 (1997).
6. Zoratti, M. and Szabo, In. The mitochondrial permeability transition. Biochim. Biophys. Acta 1241, 139-76 (1995).
7. Stepien, G., Torroni, A., Chung, A. B., Hodge, J. A. and Wallace, D.C. Differential expression of adenine nucleotide translocator isoforms in mammalian tissues and during muscle cell differentiation. J. Biol. Chem. 267, 14592-7 (1992).
8. Lunardi, J., Hurko, O., Engel, W. K. and Attardi, G. The multiple ADP/ATP translocase genes are differentially expressed during human muscle development. J. Biol. Chem. 267, 15267-70. (1992).
9. Levy, S. E., Chen, Y. S., Graham, B. H. and Wallace, D.C. Expression and sequence analysis of the mouse adenine nucleotide translocase 1 and 2 genes. Gene 254, 57-66. (2000).
10. Ellison, J. W., Salido, E. C. and Shapiro, L. J. Genetic mapping of the adenine nucleotide translocase-2 gene (Ant2) to the mouse proximal X chromosome. Genomics 36, 369-71.
(1996).11. Ceci, J. D. Mouse chromosome 8. Mamm. Genome 5, S124-38. (1994).
12. Rial, E. et al. Retinoids activate proton transport by the uncoupling proteins UCP1 and UCP2. EMBO J. 18, 5827-33. (1999).
13. Boss, O., Hagen, T. and Lowell, B. B. Uncoupling proteins 2 and 3: potential regulators of mitochondrial energy metabolism. Diabetes 49, 143-56. (2000).
14. Marzo, In. et al. Bax and adenine nucleotide translocator cooperate in the mitochondrial control of apoptosis. Science 281, 2027-31 (1998).
15. Petronilli, V., Cola, C., Massari, S., Colonna, R. and Bernardi, P. Physiological effectors modify voltage sensing by the cyclosporin A-sensitive permeability transition pore of mitochondria. J. Biol. Chem. 268, 21939-45 (1993).
16. Lapidus, R. G. and Sokolove, P. M. The mitochondrial permeability transition. Interactions of spermine, ADP, and inorganic phosphate. J. Biol. Chem. 269, 18931-6 (1994).
17. Bernardi, P. Modulation of the mitochondrial cyclosporin A-sensitive permeability transition pore by the proton electrochemical gradient. Evidence that the pore can be opened by membrane depolarization. J. Biol. Chem. 267, 8834-9. (1992).
8. Di Lisa, F. and Bernardi, P. Mitochondrial function as a determinant of recovery or death in cell response to injury. Mol. Cell. Biochem. 184, 379-91. (1998).
19. Lorenzo, H. K., Susin, S. A., Penninger, J. and Kroemer, G. Apoptosis inducing factor (AIF): a phylogenetically old, caspase-independent effector of cell death. Cell Death Differ. 6, 516-24. (1999).
20. Du, C., Fang, M., Li, Y., Li, L. and Wang, X. Smac, a mitochondrial protein that promotes cytochrome c-dependent caspase activation by eliminating IAP inhibition. Cell 102, 33-42. (2000).
21. Verhagen, A. M. et al. Identification of DIABLO, a mammalian protein that promotes apoptosis by binding to and antagonizing IAP proteins. Cell 102, 43-53. (2000).
22. Susin, S. A. et al. Mitochondrial release of caspase-2 and -9 during the apoptotic process. J. Exp. Med 0.189, 381-94. (1999).
23. Zamzami, N. and Kroemer, G. The mitochondrion in apoptosis: how Pandora's box opens. Nat. Rev. Mol. Cell Biol. 2, 67-71. (2001).
24. Martinou, J. C. and Green, D. R. Breaking the mitochondrial barrier. Nat. Rev. Mol. Cell Biol. 2, 63-7. (2001).
25. Graham, B. H. et al. A mouse model for mitochondrial myopathy and cardiomyopathy resulting from a deficiency in the heart/muscle isoform of the adenine nucleotide translocator. Nat. Genet. 16, 226-34 (1997).
26. Copp, A. J. Death before birth: clues from gene knockouts and mutations. Trends Genet. 11, 87-93. (1995).
27. Rossant, J. Mouse mutants and cardiac development: new molecular insights into cardiogenesis. Circ. Res. 78, 349-53. (1996).
28. Fontaine, E., Eriksson, O., Ichas, F. and Bemardi, P. Regulation of the permeability transition pore in skeletal muscle mitochondria. Modulation By electron flow through the respiratory chain complex in. J. Biol. Chem. 12662-8 (1998).
29. Postic, C. and Magnuson, M. A. DNA excision in liver by an albumin-Cre transgene occurs progressively with age. Genesis 26, 149-50. (2000).
30. Petronilli, V., Nicolli, A., Costantini, P., Colonna, R. and Bernardi, P. Regulation of the permeability transition pore, a voltage-dependent mitochondrial channel inhibited by cyclosporin A. Biochim. Biophys. Acta 1187, 255-9 (1994).
31. Novgorodov, S. A., Gudz, T. In., Brierley, G. P. and Pfeiffer, D. R. Magnesium ion modulates the sensitivity of the mitochondrial permeability transition pore to cyclosporin A and ADP. Arch. Biochem. Biophys. 311, 219-28 (1994).
32. Kaukonen, J. et al. Role of adenine nucleotide translocator 1 in mtDNA maintenance. Science 289, 782-5. (2000).
33. Esposito, L. A., Melov, S., Panov, A., Cottrell, B. A. and Wallace, D.C. Mitochondrial disease in mouse results in increased oxidative stress. Proc. Natl. Acad. Sci. USA 96, 4820-5. (1999).
34. Arnold, R. S. et al. Hydrogen peroxide mediates the cell growth and transformation caused by the mitogenic oxidase Nox1. Proc. Natl. Acad. Sci. USA 98, 5550-5. (2001).
35. Burdon, R. H. Superoxide and hydrogen peroxide in relation to mammalian cell proliferation. Free Radic. Biol. Med. 18, 775-94. (1995).
36. Yeh, W. C. et al. FADD: essential for embryo development and signaling from some, but not all, inducers of apoptosis. Science 279, 1954-8. (1998).
37. Varfolomeev, E. E. et al. Targeted disruption of the mouse Caspase 8 gene ablates cell death induction by the TNF receptors, Fas/Apol, and DR3 and is lethal prenatally. Immunity 9, 267-76. (1998).
38. Vyssokikh, M. Y. et al. Adenine nucleotide translocator isoforms 1 and 2 are differently distributed in the mitochondrial inner membrane and have distinct affinities to cyclophilin D. Biochem. J. 358, 349-58. (2001).
39. Wilkinson, D. G. and Nieto, M. A. Detection of messenger RNA by in situ hybridization to tissue sections and whole mounts. Meth. Enzymol. 225, 361-73 (1993).
40. Ross, A. J., Amy, S. P., Mahar, P. L., Lindsten, T., Knudson, C. M., Thompson, C. B., Korsmeyer, S. J., and MacGregor, G. R. BCLW Mediates Survival of Postmitotic Sertoli Cells by Regulating BAX Activity. Developmental Biology 239, 295-308 (2001).
41. Ellison, J. W., Li, X., Francke, U. and Shapiro, L. J. Rapid evolution of human pseudoautosomal genes and their mouse homologs. Mamm. Genome 7, 25-30. (1996).
42. Trounce, I. A., Kim, Y. L., Jun, A. S. and Wallace, D.C. Assessment of mitochondrial oxidative phosphorylation in patient muscle biopsies, lymphoblasts, and transmitochondrial cell lines. Meth. Enzymol. 264:484-509 (1996).
43. Kokoszka, J. E., Coskun, P., Esposito, L. A. and Wallace, D.C. Increased mitochondrial oxidative stress in the Sod2 (+/−) mouse results in the age-related decline of mitochondrial function culminating in increased apoptosis. Proc. Natl. Acad. Sci. USA 98, 2278-83. (2001).
44. Esposito, L. A. et al. Mitochondrial oxidative stress in mice lacking the glutathione peroxidase-1 gene. Free Radic. Biol. Med. 28, 754-66. (2000).
45. Demura, M., Kamo, N. and Kobatake, Y. Mitochondrial membrane potential estimated with the correction of probe binding. Biochim. Biophys. Acta 894, 355-364 (1987).
46. Kamo, N., Muratsugu, M., Hongoh, R. and Kobatake, Y. Membrane potential of mitochondria measured with an electrode sensitive to tetraphenyl phosphonium and relationship between proton electrochemical potential and phosphorylation potential in steady state. J. Membr. Biol. 49, 105-21. (1979).
47. Rottenberg, H. Membrane potential and surface potential in mitochondria: Uptake and binding of lipophilic cations. J. Membr. Biol. 81, 127-38 (1984).
48. U.S. Pat. No. 6,013,858 (Wallace et al., 2000).
49. Qian, T., Herman, B., and Lemasters, J. J. The mitochondrial permeability transition mediates both necrotic and apoptotic death of hepatocytes exposed to Br-A23187. Toxicol. Appl. Pharm. 154: 117-125 (1999).
50. Leist M., Gantner F., Bohlinger I., Germann, P. G., Tiegs G. and Wendel A. Murine hepatocyte apoptosis induced in vitro and in vivo by TNF-□ requires transcriptional arrest. J. Immunol. 153:1778-1788 (1994).
51. Hatono, E., Bradham, C. A., Stark, A., Iimuro, Y., Lemasters, J. J., and Brenner, D. A. The mitochondrial permeability transition augments Fas-induced apoptosis in mouse hepatocytes. J. Biol. Chem. 275: 11814-11823 (2000).

1. A transgenic mouse in which an Ant2 gene, which encodes a systemic adenine nucleotide translocator isoform, is inactivated in a tissue-specific fashion.

2. The transgenic mouse of claim 1, wherein a heart-muscle adenine nucleotide translocator (Ant1) is inactivated.

3. The transgenic mouse of claim 1, wherein the Ant2 gene is inactivated in liver.

4. The transgenic mouse of claim 2, wherein the Ant2 gene is inactivated in liver.

5. Liver, hepatocytes, or mitochondria or mitochondrial fractions thereof in which an Ant2 gene encoding a systemic isoform of adenine nucleotide translocator is inactivated.

6. The liver, hepatocytes, or mitochondria or mitochondrial fractions thereof of claims 5, in which an Ant1 gene encoding a heart-muscle isoform of adenine nucleotide translocator is inactivated.

7. A method for assessing sensitivity of Ant2-deficient animals or humans to a test composition or condition, said method comprising the steps of contacting a test compound or an environmental condition with liver, hepatocytes, mitochondria, mitochondrial membranes lacking a functional Ant2 gene product and measuring viability, membrane potential or the mitochondrial permeability transition pore (mtPTP) as compared to measurements taken with liver, hepatocytes, mitochondria or mitochondrial membranes which express a functional Ant2 gene product.

8. A method for identifying a test composition or environmental condition which alter the function of a mitochondrial adenine nucleotide translocator in an animal or human lacking a functional Ant2 gene product as compared with an animal or human expressing a functional Ant2 gene product, comprising the steps of
(a) providing a mouse which lack a functional Ant2 gene product in one or more tissues of interest;
(b) contacting the mouse of step (a) or cells, mitochondria, proteins or subcellular components thereof with the test composition or condition;
(c) comparing at least one response of the mouse of step (a) or cells, mitochondria, proteins or subcellular components thereof as compared to a normal mouse, or cells, mitochondria, or subcellular component thereof; and
(d) identifying a test composition or environmental conditions when the contacting of step (b) alters the response measured, in the mouse of step (a) as compared with a normal mouse.
